(19) United States
(12) Patent Application Publication
(10) Pub. No.: US 2025/0053848 A1
(43) Pub. Date: Feb. 13, 2025

(54) METHOD AND SYSTEM FOR ESTIMATING A GROUND STATE ENERGY OF A QUANTUM CHEMICAL SYSTEM

(71) Applicants: Volkswagen Aktiengesellschaft, Wolfsburg (DE); XANADU Quantum Technologies Inc., Toronto (CA)

(72) Inventors: Juan Miguel Arrazola Mantilla, Toronto (CA); Kasra Hejazi, Los Angeles, CA (US); Modjtaba Shokrian Zini, Toronto (CA); Stepan Fomichev, Toronto (CA); Matthew Royal Kiser, München (DE)

(21) Appl. No.: 18/789,483

(22) Filed: Jul. 30, 2024

(30) Foreign Application Priority Data
Aug. 4, 2023 (EP) 23189718.2

(51) Int. Cl. G06N 10/70

(52) U.S. Cl. CPC G06N 10/70

(57) ABSTRACT

Technologies and techniques for estimating the ground state energy of a quantum chemical system. The method includes classically computing states close to the ground state of the quantum chemical system, assessing the quality of the classically computed states based on their energy distribution, selecting a state based on the assessment result, implementing the selected state on a quantum computer, and estimating the ground state energy of the quantum chemical system on the quantum computer based on the implemented state. The disclosure also encompasses various technologies and techniques for preparing a quantum state for quantum energy estimation on a quantum chemical system and a system for performing these methods.

### RELATED APPLICATIONS

The present application claims priority to European Patent Application No. 23 189 718.2, to Arrazola Mantilla et al., filed on Aug. 4, 2023, the contents of which are incorporated by reference in their entirety herein.

### TECHNICAL FIELD

The present disclosure is directed to technologies and techniques for estimating a ground state energy of a quantum chemical system and a system. Further, the present disclosure relates to technologies and techniques for preparing a quantum state for quantum energy estimation on a quantum chemical system.

### BACKGROUND

There is significant interest in obtaining accurate estimates of the ground state energies of electronic systems, due to their relevance to practical applications such as the design of new chemical reactions, catalysts, materials, and more. Preparing an initial state with sufficient quality is essential for current methods of quantum energy estimation. Conventionally, energy estimation is considered for the ground states of many-body systems, and the quality of the initial state is assessed by its overlap with the ground state of the system. Methods ranging from quantum phase estimation and its variants to more recent developments all require a lower bound on the overlap with the target state whose energy is being estimated.

In all these schemes, the lower bound on the overlap also appears directly in the cost, usually through the factor 1/overlap² (or 1/overlap when a method such as amplitude amplification is used), which determines the number of repetitions required to obtain the energy of the target state.

This highlights the importance of preparing a high-quality initial state for the efficiency and relevance of the entire quantum energy estimation procedure. Regarding practical state preparation, many different methods may be considered. These range from quantum heuristic algorithms such as adiabatic state preparation, variational methods, quantum imaginary time evolution, to quantum preparation of a classically computed ansatz. The latter can take many forms based on the classical ansatz considered for implementation, such as Hartree-Fock, (unitary) coupled cluster, sums of Slater determinants, matrix product states, and more. However, despite the variety of possible classical ansatzes, most prior art focuses on implementing Hartree-Fock states, which are known to be poor representations for strongly correlated many-body systems—the main candidates for useful application of quantum energy estimation routines. Thus, there is a demand for improvement.

The present disclosure addresses the technical problem of improving a method and system for estimating the ground state energy of a quantum chemical system.

### SUMMARY

Aspects of the present disclosure are described in the features recited in the independent claims, found below. Further aspects are described in the features recited in the dependent claims.

In some examples, a method is provided for estimating a ground state energy of a quantum chemical system. The method involves classically computing states close to the ground state of the quantum chemical system, assessing the quality of these classically computed states based on their energy distribution, selecting a state based on this assessment, implementing the selected state on a quantum computer, and estimating the ground state energy of the quantum chemical system on the quantum computer based on the implemented state.

Additionally, a system is provided comprising a classical computer and a quantum computer. The classical computer is configured to perform the classical computations of a method described in any one of the embodiments of this disclosure, and the quantum computer is configured to perform the quantum computations of the method.

In a second aspect, a method for preparing a quantum state for quantum energy estimation on a quantum chemical system is provided. This method includes classically computing states close to the ground state of the quantum chemical system, assessing the quality of these classically computed states based on their energy distribution, selecting a state based on this assessment, and implementing the selected state on a quantum computer. The advantages and details of this aspect are the same as those described for the respective features above and below.

In some examples, implementing the selected state involves preparing and using a compressed representation of the sum of Slater determinants. The advantages and details of this example are consistent with those described for the respective features above and below.

In some examples, the method further includes quantum refining of the implemented state on the quantum computer by filtering out higher energy contributions after state implementation. The advantages and details of this example are consistent with those described for the respective features above and below.

In a third aspect, a method for preparing a quantum state for quantum energy estimation on a quantum chemical system is provided. This method involves classically computing a state close to the ground state of the quantum chemical system and implementing the state on a quantum computer by preparing and using a compressed representation of the sum of Slater determinants. The advantages and details of this aspect are consistent with those described for the respective features above and below.

In some examples, classically computing the state involves computing a plurality of states close to the ground state of the quantum chemical system, assessing the quality of these classically computed states based on their energy distribution, and selecting a state based on this assessment for implementation. The advantages and details of this example are consistent with those described for the respective features above and below.

In some examples, the method further includes quantum refining of the implemented state on the quantum computer by filtering out higher energy contributions after state implementation. The advantages and details of this example are consistent with those described for the respective features above and below.

In a fourth aspect, a method for preparing a quantum state for quantum energy estimation on a quantum chemical system is provided. This method includes classically computing a state close to the ground state of the quantum chemical system, implementing the state on a quantum computer, and refining the implemented state on the quantum computer by filtering out higher energy contributions after state implementation. The advantages and details of this aspect are consistent with those described for the respective features above and below.

In some examples, classically computing the state involves computing a plurality of states close to the ground state of the quantum chemical system, assessing the quality of these classically computed states based on their energy distribution, and selecting a state based on this assessment for implementation. The advantages and details of this example are consistent with those described for the respective features above and below.

In some examples, preparing the state involves preparing and using a compressed representation of the sum of Slater determinants. The advantages and details of this example are consistent with those described for the respective features above and below.

In some examples, the method further includes categorizing the ground state energy estimation problem of the quantum chemical system with regard to quantum computational difficulty as either an easy problem, an intermediate problem, or a hard problem. If the result shows an intermediate problem, all method steps are performed. This allows the method to be used only when specific conditions are met, i.e., when the method is advantageous in terms of cost (computational/state preparation cost) relative to classical method costs. The idea is that with a given budget for state preparation, one can have easy, intermediate, and hard problems. An easy problem is one where a very high-quality initial state with large support on the low-energy subspace of the Hamiltonian can be prepared on a quantum computer. A hard problem is one where, with a given budget, only a poor-quality state with negligible weight over low energies of the Hamiltonian can be prepared. Intermediate problems are those where there is some non-negligible, but not close to 1, weight over low-energy parts of the spectrum.

For intermediate problems, it is possible to obtain a quantum advantage in quantum ground state energy determination over classical computational methods. This is because, by definition, for hard problems one cannot perform quantum routines effectively, and for easy problems, it is very likely to find a good classical energy estimate which is difficult to surpass with quantum methods

In some examples, the method further comprises calculating, based on the energy distribution, the mean value of the distribution of the smallest energy obtained from within a quantum phase estimation budget of N outcomes, assessing whether it is below a target classical energy, and only if this is the case, performing all method steps. This provides a criterion for deciding if the method should be performed. The example and the criterion are illustrated as follows: The energy distribution is needed. Let the best classically achieved energy be denoted by E_{T}. Assuming access to the energy distribution of the initial state, one can calculate the distribution of the best energy achievable through N QPE measurements within the budget and evaluate its mean value. If the mean value is below the classical target energy E_{T}, this suggests the possibility for quantum advantage. Thus, there is potential for improving the ground energy estimate using QPE.

### BRIEF DESCRIPTION OF THE DRAWINGS

Aspects of the present disclosure are explained in further detail below on the basis of preferred exemplary embodiments with reference to the drawings. In the drawings:

FIG. 1 shows a schematic diagram to illustrate embodiments of a method for estimating a ground state energy of a quantum chemical system, according to some aspects of the present disclosure;

FIG. 2 shows a schematic diagram to illustrate embodiments of the method for preparing a quantum state for quantum energy estimation on a quantum chemical system, according to some aspects of the present disclosure;

FIG. 3 shows a schematic diagram to illustrate embodiments of the method for preparing a quantum state for quantum energy estimation on a quantum chemical system, according to some aspects of the present disclosure;

FIG. 4 shows a schematic diagram to illustrate embodiments of the method for preparing a quantum state for quantum energy estimation on a quantum chemical system, according to some aspects of the present disclosure; and

FIG. 5 shows a schematic of an embodiment of the system, according to some aspects of the present disclosure.

### DETAILED DESCRIPTION

One of the main ideas of the present disclosure is to compute, using classical (non-quantum) methods on a classical computer, candidate states close to the ground state of the quantum chemical system for which the ground state energy is to be estimated. After calculating various candidates for later implementation on the quantum computer, the most suitable candidate is selected. To this end, the quality of each classically computed state is assessed based on the energy distribution of the respective state. The best candidate is selected based on this assessment. With the energy-distribution- based quality assessment technique, it is possible to decide between different classically found candidates and choose the one with the highest quality. The selected state is implemented on the quantum computer, and the ground state energy of the quantum chemical system is estimated on the quantum computer based on the implemented state. In other words, the selected state is used as the initial state that is propagated through the circuit of the quantum computer to find the ground state energy.

The present disclosure is based on the finding that the conventional criterion, i.e., evaluating the overlap with the ground state, is a difficult task and is often in conflict with the purpose of performing quantum energy estimation for the given problem, as it requires substantial knowledge about the ground state of the system. It is preferable to assess the quality of the candidate states based on the full energy distribution because, unlike the ground state overlap, there are ways to obtain approximated energy distributions both classically and quantumly. Furthermore, sufficient information for assessing the quality of the state can be obtained from the energy distribution. The accumulated weight of the energy distribution for low energies, also referred to as the support on the low- energy subspace, is an important quantity to consider as it is proportional to the probability of obtaining low energy estimates in a quantum routine. The candidate state is chosen that has the largest weights for smaller energies.

To calculate the states close to the ground state of the quantum chemical system, a Hamiltonian describing the system is defined, and a traditional quantum chemistry method is executed on a classical computer to obtain an approximate solution for the ground state. The classical computation is performed to obtain an initial state that potentially has large support on the low-energy subspace of the Hamiltonian. Different classical methods may be used for this calculation, such as Hartree-Fock, Configuration Interaction with singles and doubles (CISD), Coupled Cluster (CC) with single or double excitations (CCSD), complete active space configuration interaction (CASCI), multireference perturbation theory (MRPT), selective configuration interaction methods like semi-stochastic heat bath configuration interaction (SHCI), and tensor network ansatz methods, in particular, density matrix renormalization group (DMRG).

Finally, the ground state energy estimated by the method described in this disclosure is provided. A signal and/or data describing the estimated ground state energy is produced.

### Energy Distribution

In the following, the procedure for deriving and estimating the energy distribution is described. The energy distribution of a candidate initial state |ψ [symbol] with respect to the Hamiltonian H may be defined as follows:

:

$$A ( ω ) = {\sum }_{E} {\vert 〈 E \vert ψ 〉 \vert}^{2} {f}_{η} ( E - ω ) \qquad ( Eq . 1 )$$

where E, |E [symbol] are eigenvalues and eigenstates of H and f_{η} is some kernel, e.g., Gaussian, Lorentzian, etc., with width η centered at the position of each of the eigenvalues. η=0 corresponds to a discrete distribution, the actual energy distribution of the state, while with η≠0 each energy value is broadened and the result is a continuous distribution. In practice, it will be hard to access the true η=0 distribution and focus should thus be on broadened versions.

A simple criterion based on the energy distribution can be formulated for the quality of states for performing energy estimation; supposing a number of candidate initial states are given with similar energies, i.e., with similar expectation values of H, the quality can be compared by focusing on their distribution and in particular on their left-side (i.e., low-energy-side) tails. Roughly, whichever state that has more weight extended to lower energies is a better candidate as it provides higher probability for obtaining a low energy estimate in the energy estimation process.

Assessing the quality involves, for each potential classically computed candidate state, calculating a distribution of QPE outcomes of N measurements based on the energy distribution of the potential candidate state, calculating a cumulative distribution function of the minimum energy of each QPE outcome with respect to a reference energy Eₒ, differentiating the cumulative distribution function with respect to the reference energy Eₒ, and determining the properties of the lowest possible outcome based on the differentiated cumulative distribution function. The lowest possible outcomes of the candidate states are compared with the goal of choosing the smallest one.

Selecting a state may involve using the mean value of the differentiated distribution and choosing the candidate state with the smallest mean value.

### Using Energy Distribution for Estimating Lowest Outcomes

Assuming access to the (approximate) energy distribution of the candidate state of interest, the distribution of QPE outcomes can be calculated as follows for k digits and an integer outcome xₒ:

$${\tilde{A}}_{QPE} ( {x}_{o} ) = \int dE A ( E ) \frac{1}{{2}^{2 k}} ( \frac{{sin}^{2} ( π {2}^{k} E )}{{sin}^{2} ( π [ E - {x}_{o} / {2}^{k} ] )} )$$

This is a discrete distribution function. The distribution of potential QPE outcomes and, in particular, the lowest possible measurements that are expected to be obtained are studied. Since high-precision QPE is performed and for simplicity of presentation, an approximation is used, and the continuous limit of the outcome distribution with Eₒ=2^{-k}xₒ is taken. Then defining A_{QPE}(Eₒ)=2^{-k}Ã_{QPE}(xₒ), it is noted that in the limit of k→∞, A_{QPE} approaches A and thus A(E) is used in the following discussion. It is straightforward to undo this and work with the actual discrete QPE outcome distribution when needed.

To understand the statistics of the best energy achievable through a number of QPE measurements N, suppose the QPE outcomes are E₁, . . . , E_{N}. The distribution of Eₘᵢₙ^{(N)}=min(E₁, . . . , E_{N}) is considered. The cumulative distribution function of this variable at energy E₀ as:

$${C}_{{min}_{N}} ( {E}_{0} ) = 1 - {( 1 - p < ( {E}_{0} ) )}^{N} \qquad ( Eq . 3 )$$

This is the probability that at least one outcome from N rounds of QPE lies below E₀: here p<(Eₒ)=∫_{-∞}^{E}^{₀}dE A(E) is the probability of a single outcome lying below E₀. In this way, one can see that Cₘᵢₙ_{_{N}} is also the probability that the smallest outcome from the list E₁, . . . , E_{N} is below E₀: either there is only one, and it by definition the smallest, or there are several and then one of them is the smallest-but they are all definitely below E₀. Upon differentiating the cumulative distribution function with respect to E₀, one obtains the probability distribution function of min (E₁, . . . , E_{N}), which reads:

$${A}_{min}^{( N )} ( {E}_{0} ) = NA ( {E}_{0} ) {( 1 - p < ( {E}_{0} ) )}^{N - 1}$$

With this probability distribution function, one can determine the properties of the lowest possible outcome from N QPE measurements, and in doing so, compare different candidate states associated with these distributions and choose the best one. In particular, one simple measure of state quality through this approach may be the mean value of this distribution: ∫dEAₘᵢₙ^{(N)}(E) E. A state whose associated mean value is smaller is judged to be better suited for running QPE.

Another way of understanding the statistics of the lowest outcome from N rounds of QPE measurements is as follows. Consider again the probability of the smallest of N outcomes lying below E₀ shown in Eq. (3). Suppose one would like to find the threshold E₀, beyond which this probability falls below 1−δ, with 0<δ<0.5, and take this threshold as a measure of how small of an outcome is probable for a given energy distribution. This threshold can be found as the following; we find the E₀such that the following equations hold:

$${C}_{{min}_{N}} ( {E}_{0} ) = 1 - δ \qquad ( Eq . 4 )$$

$${( 1 - p < ( {E}_{0} ) )}^{N} = δ$$

$$N = log δ / log [ 1 - p < ( {E}_{0} ) ]$$

For large N, one expects p< (E₀) to be small and thus Taylor expansion of the logarithm results in

$$N = 𝒪 ( log ( \frac{1}{δ} ) \frac{1}{p < ( {E}_{0} )} ) .$$

Solving the last row of Eq. (4) for E₀ gives the desired threshold beyond which the probability of obtaining an outcome becomes smaller than 1−δ.

Now, the quality of two energy distributions A₁ and A₂ may be compared; two methods are discussed for this. First, to this end, the probability of obtaining a lower outcome from distribution A₂ is considered, which can be written as:

$${I}_{1 ; 2} = {\int }_{- ∞}^{∞} {dE}_{1} {A}_{1 , min}^{( N )} ( {E}_{1} ) {\int }_{- ∞}^{{E}_{1}} {dE}_{2} {A}_{2 , min}^{( N )} ( {E}_{2} )$$

Depending on whether I_{1;2} is larger or smaller than ½, A₂ or A₁ is more capable of producing lower energies with N QPE measurements.

Another simple criterion can be given by the threshold obtained from Eq. (4). With a choice of δ, whichever probability distribution gives a smaller threshold can be chosen as the higher quality one.

In one embodiment, the method further comprises approximating the energy distributions of at least a subset of the classically computed states through an Edgeworth series expansion using the Hamiltonian moments of the state and/or by the resolvent (matrix product state-based) method. These are classical methods which are implemented on the classical computer.

### Edgeworth Series

For the Edgeworth series, the moments of energy (expectation values of powers of H) are used and series expansions are obtained for the energy distribution. The Edgeworth series and the Gram-Charlier series are considered for illustration. Both approximate a distribution as a Gaussian multiplied by different orders of the Hermite polynomials. The coefficients of the terms in the series, can be written in terms of the moments of the distribution, or in other words the expectation values of powers of the Hamiltonian [symbol] ψ|Hⁿ|ψ [symbol] . The lowest order approximation in this scheme is a Gaussian distribution with a variance proportional to [symbol] ψ|H²|ψ [symbol] . It is noted that this method works best for distributions that are close to Gaussian. In the following, these series expansion techniques are discussed.

The Edgeworth and Gram-Charlier series have identical terms, the only difference being that the terms in an Edgeworth series are arranged in a way that the series constitutes a true asymptotic series [12]. First, details on the Gram-Charlier series are given; the expansion can be written as:

$${P}_{GS} ( x ) = \frac{{e}^{- {x}^{2} / 2}}{\sqrt{2 π}} [ 1 + {\sum }_{n = 3}^{∞} {( - 1 )}^{n} {c}_{n} {He}_{n} ( x ) ] \qquad ( Eq . 5 )$$

where Heₙ(x) is the Hermite polynomial in the probabilist's notation defined as:

$${He}_{n} ( x ) = {( - 1 )}^{n} {e}^{\frac{{x}^{2}}{2}} \frac{{d}^{n}}{{dx}^{n}} {e}^{- \frac{{x}^{2}}{2}}$$

$$\int dx \frac{{e}^{- {x}^{2} / 2}}{\sqrt{2 π}} {He}_{n} ( x ) {He}_{m} ( x ) = {δ}_{nm} n !$$

The expansion in the form in Eq. (5) is used for a distribution function with zero mean and unit variance; it is noted that any distribution satisfies these two criteria upon translating and rescaling.

Using the above orthonormality relations, one is able to determine the coefficients of the Gram-Charlier expansion for a distribution function p(x) order by order as follows:

$${c}_{n} = \frac{{( - 1 )}^{n}}{n !} \int dx p ( x ) {He}_{n} ( x )$$

It is clear that the coefficients can be written in terms of the moments of the distribution μₙ=∫ dx p(x)xⁿ. A list of the coefficients is given in Table 1.

TABLE 1
Coefficients cₙ defined in the equation mentioned above.

| order | Gram-Charlier coefficient |
|---|---|
| 3 | $- \frac{1}{3 !} {μ}_{3}$ |
| 4 | $\frac{1}{4 !} [ {μ}_{4} - 3 ]$ |
| 5 | $\frac{1}{5 !} [ - {μ}_{5} + 10 {μ}_{3} ]$ |
| 6 | $\frac{1}{6 !} [ {μ}_{6} - 15 {μ}_{4} + 30 ]$ |
| 7 | $\frac{1}{7 !} [ - {μ}_{7} - 21 {μ}_{5} - 105 {μ}_{3} ]$ |
| 8 | $\frac{1}{8 !} [ {μ}_{8} - 28 {μ}_{6} + 210 {μ}_{4} - 315 ]$ |

On the other hand, the Edgeworth series as mentioned above is an asymptotic series that is obtained by grouping the same terms in a Gram-Charlier series in a particular way:

$${P}_{E} ( x ) = \frac{{e}^{- {x}^{2} / 2}}{\sqrt{2 π}} [ 1 + \frac{{κ}_{3}}{6} {He}_{3} ( x ) + ( \frac{{κ}_{4}}{24} {He}_{4} ( x ) + \frac{{κ}_{3}^{2}}{72} {He}_{6} ( x ) ) + ( \frac{{κ}_{5}}{120} {He}_{5} ( x ) + \frac{{κ}_{4} {κ}_{3}}{144} {He}_{7} ( x ) + \frac{{κ}_{3}^{3}}{1296} {He}_{9} ( x ) ) + … ]$$

where κₙ is the nth cumulant of the distribution:

$${κ}_{3} = {μ}_{3}$$

$${κ}_{4} = {μ}_{4} - 3$$

$${κ}_{5} = {μ}_{5} - 10 {μ}_{3}$$

$$…$$

For the general prescription for obtaining the terms and explicit forms for more terms, see Appendix A. In practice, in some cases, the above series expansions exhibit irregular behaviors like having negative values, artificial rapid oscillations, etc. This happens mostly in cases where the energy distribution is very far from a Gaussian. Large gaps in the spectrum can also result in such behavior.

Generally, the series will converge if the approximated distribution functions falls faster than exp(−x²/4) for large x [12]. Even if the above series do not converge, it is still possible to truncate them, particularly the Edgeworth series as it is an asymptotic series, and still obtain some useful information. The convergence condition is satisfied for a bounded energy spectrum. However, the discreteness of the true energy distribution can be problematic for the approximation. Here, focus is set on computation of moments of energy through the matrix product state (MPS) language. This should not impose a large computational overhead as acting with the Hamiltonian matrix product operator on the solution MPS even multiple times is not a prohibitive task. Particularly, in practice it can be seen that it is simple to go moments of order ˜10 without a need to increase the state's bond dimension substantially. The lowest order approximation in the series corresponds to a Gaussian fitting to the distribution and the only information that is required for it is the variance of energy in the initial state [symbol] ψ|H²|ψ [symbol] .

### Resolvent Method

The other classical approach is the resolvent method. This is a classical computational method in which the goal is to find the Green's function defined as below:

$$G ( ω , η ) = \frac{1}{π} 〈 ψ \vert \frac{1}{H - ω + i η} \vert ψ 〉$$

where η is a broadening factor. From this Green's function, one can get the energy distribution function for energy ω as the following:

$$A ( ω , η ) = - Im G ( ω , η )$$

It is noted that since

$$Im \frac{1}{E - ω + i η} = \frac{- η}{{( E - ω )}^{2} + {η}^{2}}$$

this method amounts to using a Lorentzian kernel with broadening η in Eq. (1).

The above Green's function is solved for through a DMRG-like variational method that was introduced in Ref. [13] and then improved in Ref. [14]. The former method is called dynamical DMRG (DDMRG) and the improved version is called DDMRG++ in Ref. [14]. It is an MPS method and performs DMRG-like sweeps to evaluate the above Green's function. This means that the state that is to be implemented on the quantum computer needs to be transformed into MPS form, which is possible as discussed earlier.

The method works as follows; the state |φ [symbol] is defined that satisfies:

$$\vert ϕ 〉 = \frac{1}{π} \frac{1}{( H - ω + i η )} \vert ψ 〉 ⇒ π ( H - ω + i η ) \vert ϕ 〉 = \vert ψ 〉$$

The above Green's function can be written as the overlap G(ω, η)= [symbol] |φ [symbol] . Now, defining |Y [symbol] as [13]:

$$[ {( H - ω )}^{2} + {η}^{2} ] \vert Y 〉 = - \frac{η}{π} \vert ψ 〉$$

It is easy to see that Im G= [symbol] ω|Y [symbol] . Finding the overlap of the above equation with |Y [symbol] , a DMRG-like algorithm is used to minimize the resulting functional [13, 14]:

$$〈 Y \vert [ {( H - ω )}^{2} + {η}^{2} ] \vert Y 〉 + \frac{η}{π} 〈 Y \vert ψ 〉$$

For example, the package block2 [6] may be used for implementation of DDMRG++. Even though the method is implemented in the MPS language, it can be thought of as general because as discussed in Appendix B, there are methods for transforming other forms of states into MPS. It is also mentioned that the same Green's function can be calculated using time evolution, namely calculating the overlap [symbol] ψ|e^{iHt}|ψ [symbol] and then Fourier transforming (with the addition of a damping function e^{-ηt}). Similar MPS-based methods can also be used to this end [14].

In some examples, the method includes approximating the energy distributions of at least a subset of the classically computed states through coarse quantum phase estimation (QPE). It is noted that the classically computed states must be implemented on a quantum computer in order to perform coarse QPE. With a quantum computer capable of performing precision energy estimation, it is expected to also be able to perform lower precision QPE. Performing lower precision (coarse) QPE can give an approximate energy distribution function. In the following, a method that approximates an energy distribution in the form of Eq. (1) with f_{η} given by the QPE kernel is described:

$${K}_{QPE} ( ω ) = \frac{1}{{2}^{2 k}} \frac{{sin}^{2} ( π {2}^{k} ω )}{{sin}^{2} ( π ω )}$$

for some integer k that is the number of digits in the coarse QPE measurement. One possible way to obtain an energy distribution is through performing QPE multiple times, obtaining the statistics of the outcomes, and then trying to reconstruct the energy distribution from that. However, the outcomes are discrete and one needs to consider further manipulations to obtain an unbiased distribution function. It is noted that the energy levels themselves are also discrete. Here, in particular a method is proposed so that, as mentioned above, each energy level is broadened by the QPE kernel.

For this, performing QPE multiple times is considered, but here for each QPE round, in particular a random constant c is added to the Hamiltonian under which the QPE time evolution is performed. The constant c is chosen to lie in the interval [0, 2^{-k}) (since it is a coarse QPE measurement, these constants are not negligible). Then, after the measurement is performed and an integer xₒ is observed in the phase register of QPE, the measured energy is taken to have the value 2^{-k}xₒ−c. In this way, all real values are possible to be sampled and from performing the measurements a number of times, one is able to approximate the energy distribution using smoothing methods such as kernel density estimation [15].

In the following, a quick overview of the kernel density approximation method is given. Supposing access to a finite number of samples drawn from a distribution function is available, the goal is to approximate the distribution function. To this end, a broadening kernel is placed at the position of each of the outcomes and a normalized sum approximates the underlying distribution:

$$\hat{p} ( x ) = \frac{1}{nh} {\sum }_{i = 1}^{n} K ( \frac{x - {X}_{i}}{h} )$$

where K is a kernel (e.g., Gaussian, Lorentzian, etc.) with mean of 0 and variance of 1; Xᵢ, i=1, . . . , n are the outcomes of sampling and h is the broadening factor.

The analysis of error in reconstructing the above QPE kernel energy distribution with kernel density estimation follows a standard approach [15]. First, the error is quantified by the mean integrated square error (MISE):

$$MISE = 𝔼 ( \int {dx ( \hat{p} ( x ) - p ( x ) )}^{2} )$$

where \hat{p}(x) is the approximated distribution for the underlying distribution p(x). When a sample of size n is used, it is well known that with an appropriate choice of h, i.e., hₒₚₜ˜1/n^{1/5}, the error shows the behavior MISE˜1/n^{1/5}.

In one example, implementing the selected state comprises preparing and using a compressed representation of the sum of Slater determinants. This way a representation can be found that has a dimension smaller than that of the Hilbert space. In particular, using the compressed representation of the sum of D Slater determinants requires a Toffoli cost of [symbol] (D log D). This embodiment is particularly suited for implementing a state in a large Hilbert space, which is a sum of a number of product states much smaller than the overall Hilbert space dimension.

A simpler way to read the system register may be used by adding a smaller auxiliary register and transferring the information into the smaller auxiliary register, which is then more easily read.

In the following, this embodiment is described in more detail. In the description provided a second quantized language is used, but the implementation method is general and in particular can also be used for a first quantized implementation as well.

The normalized state Σᵢ₌₁^{D}αᵢ|α_{1,i}α_{2,i} . . . α_{2N,i} [symbol] needs to be implemented where αᵢ are the given amplitudes and α_{1,i}α_{2,i} . . . α_{2N,i} [symbol] are orbital occupation bitstrings of length 2N qubits and denote occupation of orbitals with spin ↑ or ↓. It is noted that N is the total number of orbitals. Interest is set in cases where the number of terms in the sum of Slater determinants D is much smaller than the Hilbert space dimension 2^{2N}. Therefore, the problem is to implement a summation of relatively few basis states picked from a very large Hilbert space.

Roughly, the implementation may work as follows. The aim is to implement the state

$${\sum }_{i = 1}^{D} {α}_{i} \vert {v}_{i} 〉$$

where |vᵢ [symbol] represents the orbital occupation bitstrings of length 2N mentioned above. To this end, the state Si, at li) is implemented using the quantum read-only memory (QROM) method in Ref. [8]. This will cost ˜2^{d+1} Toffoli gates for d=┌log₂(D)┐, where the enumeration register |i [symbol] has d qubits. Then a (Select or SelSwapDirty) QROM oracle O is used, of Toffoli cost 2^{d}, that reads |i [symbol] and outputs the corresponding orbital occupation bitstrings into the system register of size 2N:

$${O \vert i 〉 \vert 0 〉}^{⊗ 2 N} = \vert i 〉 \vert {v}_{i} 〉$$

which results in the following state:

$${\sum }_{i = 1}^{D} {α}_{i} \vert i 〉 \vert {v}_{i} 〉$$

Now, the enumeration register denoted by |i [symbol] in the above state needs to be uncomputed and disentangled, i.e., an operator V needs to be built such that

$${V \vert i 〉 \vert {v}_{i} 〉 = \vert 0 〉}^{⊗ d} \vert {v}_{i} 〉$$

For this, a naive approach is to use QROM one more time on the system register, i.e., |vᵢ [symbol] orbital occupation bitstrings; this will induce a cost of 2^{2N} which is exponentially large in the system size and can be prohibitive; so, an alternative method that leverages the fact that the number of terms D is much smaller than this Hilbert space dimension is used.

In particular, the following solution is used. The general idea is to efficiently construct a more compressed representation of all the Slater determinants (represented by ┌log₂ (D)┐ qubits) that can be used in the algorithm instead of the full Slater determinants of size 2N qubits. This will help reduce gate costs, as well as qubit cost, when multi-controlled CNOT gates are used.

To this end, the following matrix is considered

$${( {v}_{1} … {v}_{D} )}_{n \times D}$$

with r its rank, and where vᵢ are put as column vectors. By elementary linear algebra, a new matrix with rank r can be constructed by picking only r rows of the above matrix; in other words, there are substrings \tilde{v}ᵢ of vᵢ of length r such that

$${( {\tilde{v}}_{1} … {\tilde{v}}_{D} )}_{r \times D}$$

has rank r. Notice r≤min (n, D). The substrings \tilde{v}ᵢ in the above equation are bits at specific locations 1≤l₁< . . . <lₜ≤n of vᵢ; the indices l₁, . . . , lᵣ are identical for all vᵢ. Assuming the following lemma, it can be shown in the following corollary how the operator V can be built.

Lemma: There exists a classical algorithm with polynomial complexity in terms of (r, D), that outputs 2d−1 bitstrings uⱼ of length r, such that the bitstrings bᵢ=(u₁·\tilde{v}ᵢ, . . . , u_{2d-1}·\tilde{v}ᵢ) presented as column vectors in

$${( \begin{matrix}{u}_{1} \\ ⋮ \\ {u}_{2 d - 1}\end{matrix} )}_{( 2 d - 1 ) \times r} {( \begin{matrix}{\tilde{v}}_{1} & … & {\tilde{v}}_{D}\end{matrix} )}_{r \times D} = {( \begin{matrix}{b}_{1} & … & {b}_{D}\end{matrix} )}_{( 2 d - 1 ) \times D}$$

sare mutually distinct. The above dot products and matrix multiplication are performed modulo 2.

Corollary: Using the uᵢ in the lemma, one can build the operator V with a Toffoli cost of (2d−1)·D and additional qubit cost of 4d−3.

Proof: Recall that the state is Σᵢ₌₁^{D}αᵢ|i [symbol] |vᵢ [symbol] . Let the system register be denoted simply by v with v^{(k)} being the k-th qubit. Let |· [symbol] _{b} be an ancilla qubit initialized at |0 [symbol] . The aim is to output (u₁)₁·(\tilde{v}ᵢ)₁ inside the b register. Since (u₁)₁ is known, if it is zero, that means leaving the b register untouched, and if it is one, it means a CNOT with control on v^{(l}^{₁}⁾ and target b. One gets

$${{{\sum }_{i = 1}^{D} {α}_{i} \vert i 〉 \vert {v}_{i} 〉}_{v} \vert {( {u}_{1} )}_{1} \cdot {( {\tilde{v}}_{i} )}_{1} 〉}_{b}$$

Then with another identity map or CNOT with control on v^{(l}^{₂}⁾ and target b, the state becomes

$${{{\sum }_{i = 1}^{D} {α}_{i} \vert i 〉 \vert {v}_{i} 〉}_{v} \vert {( {u}_{1} )}_{1} \cdot {( {\tilde{v}}_{i} )}_{1} + {( {u}_{1} )}_{2} \cdot {( {\tilde{v}}_{i} )}_{2} 〉}_{b}$$

It is easy to see that repeating this process r times, delivers the state

$${{{{{\sum }_{i = 1}^{D} {α}_{i} \vert i 〉 \vert {v}_{i} 〉}_{v} \vert {u}_{1} \cdot {\tilde{v}}_{i} 〉}_{b} = {\sum }_{i = 1}^{D} {α}_{i} \vert i 〉 \vert {v}_{i} 〉}_{v} \vert {( {b}_{i} )}_{1} 〉}_{b}$$

If the register b is expanded to include 2d−1 qubits, one could do the above for all of u₂, . . . , u_{2d-1} to get the state:

$${{{\sum }_{i = 1}^{D} {α}_{i} \vert i 〉 \vert {v}_{i} 〉}_{v} \vert {( {b}_{i} )}_{1} … {( {b}_{i} )}_{2 d - 1} 〉}_{b}$$

sIt is noted that this is all done without any Toffoli so far. Rewriting, one gets

$${\sum }_{i = 1}^{D} {α}_{i} \vert i 〉 \vert {v}_{i} {〉}_{v} \vert {b}_{i} {〉}_{b}$$

Now bᵢ in register b acts as a unique small-sized flag for vᵢ. For each bᵢ, by using a multi-controlled-NOT of order 2d−1, one can flip the register i to all zero. This uses (2d−1). D Toffolis and 2d−2 additional qubits.

Of course, one also has to disentangle the register b, which is done by its uncomputation. The total Toffoli cost therefore stays at (2d−1)·D with an additional qubit cost of (2d−1)+(2d−2)=4d-3, where the term (2d−1) comes from the register b and the term (2d−2) comes from the multi-controlled-CNOTs.

In one embodiment, implementing the selected state comprises preparing and using a matrix product state (MPS) form. In particular, a state in the MPS form is implemented using an algorithm in which the matrices comprising the MPS are used directly to synthesize unitary operations required to construct the state one at a time. Direct implementation of an MPS can also impose less cost over implementing a sum of Slater determinant state in some cases. These cases are, for example, strongly correlated/multireference systems (e.g., transition-metal dimers like Cr₂/Fe₂, or clusters such as the iron-sulfur clusters [Fe₂S₂] and [Fe₄S₄] and FeMoco/nitrogenase, Hubbard models, etc.). The reason is that in such systems, it will take a large number of Slater determinants (˜10,000 or more) to encode enough of the wavefunction to get a good quality state, and the cost of Slater implementation scales with the number of determinants. In contrast, MPS can generically represent ground states of most chemical Hamiltonians of interest accurately enough with polynomial (in system size) number of parameters and cost, but translating the same MPS into a sum of Slater determinants form can result in exponentially (in system size) many parameters required.

In the following, the implementation using MPS is described in more detail. Mainly the method introduced in Ref. [4] is considered for this task and to do a Toffoli gate cost estimation. It is noted that there are many variations of this method considered in Refs. [9-11] as well as newer versions requiring lower depth circuits [5] for short range correlated MPS.

First, a quick review of the method in Ref. [4] is given. Starting with the MPS form

$$\vert ψ 〉 = {\sum }_{{n}_{1} , … {n}_{N}} {\sum }_{{α}_{1} , … , {α}_{N - 1}} {A}_{1 ; {α}_{1}}^{{n}_{1}} {A}_{2 ; {α}_{1} {α}_{2}}^{{n}_{2}} … {A}_{N ; {α}_{N - 1}}^{{n}_{N}} \vert {n}_{1} , {n}_{2} , … , {n}_{N} 〉$$

$${n}_{i} ∈ \{ v a c , ↑ , ↓ , ↑ ↓ \}$$

the standard graphical notation for manipulation of the MPS is used. Tensors are denoted as

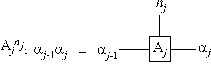

Where the physical index nⱼ runs over d values, and where d is the local Hilbert space dimension and the auxiliary indices αⱼ₋₁, αⱼ run over x values, where χ is the bond dimension. It is possible for different aⱼ's to run over different numbers, which are denoted collectively as χ above. The whole MPS is turned into a left canonical form:

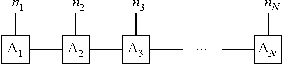

For all j>1, one has

$${\sum }_{{α}_{j} , {n}_{j}} {{A}_{j ; {α}_{j - 1} {α}_{j}}^{{n}_{j}} ( {A}_{j ; {α}_{j - 1}^{′} {α}_{j}}^{{n}_{j}} )}^{⋆} = {δ}_{{α}_{j - 1} {{α}^{′}}_{j - 1}} \qquad ( Eq . 3 )$$

or diagrammatically:

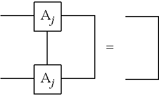

In the above equations, for the last tensor A_{N} the summation over the right auxiliary index can simply be dropped.

The method essentially works by first observing that the above tensors of the MPS, owing to the left canonical form, can be directly used to define unitaries G that are used in a quantum circuit for implementing the MPS:

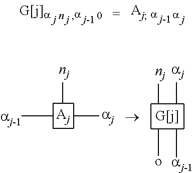

It is noted that each unitary acts on a d-level qudit or log₂(d) physical qubits and log₂ χ ancillae. For example, for fermionic systems, d=4 and two qubits are required for each o rbital of the system (each qubit can be assigned to either spin ↑ or ↓). The incoming physical index for the unitary is set to be equal to 0 and thus the above relation does not specify all the elements of G[j]. This is fine as long as the rest of the elements are chosen so that G[j] remains unitary. It is noted that Eq. (3) ensures that the specified elements of G[j] satisfy the required unitarity relations:

$${\sum }_{{α}_{j} {n}_{j}} {G [ j ]}_{{α}_{j} {n}_{j} , {α}_{j - 1} 0} {( {G [ j ]}_{{α}_{j} {n}_{j} , {{α}^{′}}_{j - 1} 0} )}^{⋆} = {δ}_{{α}_{j - 1} {{α}^{′}}_{j - 1}}$$

A quantum circuit that implements the desired MPS and works with these unitaries is shown in the schematic below. It is noted that an auxiliary register of a size denoted as [log₂ χ] is required to reconstruct the MPS and that it is schematically moved around to act with unitaries on this register and different qubits in the system. As the bond dimension can change as the circuit traverses through the system, one can start with a number of ancillae equal to [log_{2 χ}_{ₘₐₓ}] and use more or fewer of the available ancillae as the bond dimension dictates. Also, it is noted that for the first and the last unitaries the input and output auxiliary registers also have value 0.

For the above circuit one needs to synthesize the unitaries G[j] for which the unitary synthesis method of Ref. [8] may be used.

In some examples, the method further comprises quantum refining of the implemented state on the quantum computer by filtering out higher energy contributions after state implementation. This approach can decrease the cost of ground state energy estimation by reducing the number of times the most accurate quantum phase estimation (QPE) needs to be performed on the quantum computer to obtain the final result.

The aim is to filter out some of the remaining high-energy weight in the energy distribution quantumly. If the quality of the state improves, meaning the low-energy weight increases, the overall procedure becomes less costly as the number of repetitions of the most precise energy estimation algorithm is lowered. This is particularly beneficial if a cost-effective quantum refining procedure can be used.

Furthermore, the leakage problem of QPE can also be addressed using quantum refining. This means that each round of the most precise QPE measurement becomes less costly, apart from reducing the total number of repetitions. The leakage problem of QPE, which is known in the literature, changes the cost scaling of QPE to a 1/p₀² behavior instead of 1/p₀, where p₀ is the square of the overlap of the initial state

with the ground state (see I.A of [23] and Appendix A of [23]). This scaling occurs when long tails of QPE kernels placed at higher energies contribute outcomes at low energies, potentially even below the ground state value.

In principle, the leakage probability can be suppressed through the quantum refining discussed above. As the probability of leakage from low energy states themselves can be suppressed by neglecting an [symbol] (1) number of digits from QPE, the leakage mostly comes from the tail of far-away weight, i.e., from the algebraically decaying QPE kernel. If the far-away weight responsible for leakage is suppressed through quantum refining, the leakage probability is also suppressed. Identifying and filtering out the most relevant weight becomes a matter to be studied on a case-by-case basis. However, the costs for such refining can be much smaller compared to the ultimate precision QPE of interest. For example, considering an energy distribution with a multimodal structure, one can identify the peaks, with [symbol] (1) probability, responsible for leakage; then one can perform quantum refining, such as coarse QPE, to suppress those peaks after appropriate postselection. By tuning the QPE (number of digits, constants, etc.) so that the most important peaks are close to QPE outcomes that are discarded, postselection over desired outcomes will suppress these peaks. After this filtering, the probability of leakage can be reassessed, and if leakage is still probable, further quantum refining can be employed, e.g., with higher digits for the coarse QPE. Thus, the quantum refining step of the state preparation method can reduce the cost by lowering the number of required repetitions of the most precise QPE and by decreasing the cost of each round by mitigating the leakage problem.

A variety of quantum techniques may be employed for quantum refining. In some examples, quantum refining comprises using at least one of the following: Hamiltonian polynomial methods, coarse quantum phase estimation, quantum imaginary time evolution, and quantum eigenvalue transformation of unitary matrices with real polynomials (QETU).

Two of these methods are briefly described below: coarse QPE and quantum eigenvalue transformation of unitary matrices with real polynomials (QETU), where the latter is representative of the polynomial-based algorithms.

### Coarse QPE with Postselection

In this method, coarse QPE is performed, i.e., a QPE operation with fewer digits than in the ultimate-precision QPE measurement. In each coarse QPE measurement, if the outcome corresponds to high energies, the state is discarded and the quantum implementation is restarted. It is the smaller energy outcomes of QPE that one aims to postselect on; the assessment of what energies should be considered small should be addressed based on the energy distribution of the implemented state. Performing QPE on the initial state |ψ [symbol] =Σ_{E}φ_{E}|E [symbol] and then postselecting on an observed value xo, one obtains the following unnormalized state [2]:

$$\frac{1}{{2}^{k}} {\sum }_{E} {φ}_{E} \vert E 〉 \vert {x}_{o} 〉 ( \frac{1 - {e}^{2 π i {2}^{k} ( E - {x}_{o} / {2}^{k} )}}{1 - {e}^{2 π i ( E - {x}_{o} / {2}^{k} )}} )$$

meaning that the probability (=|coefficient|²) for energy E after the measurement becomes

$$\vert {φ}_{E} {\vert}^{2} \frac{1}{{2}^{2 k}} ( \frac{{sin}^{2} ( π {2}^{k} E )}{{sin}^{2} ( π [ E - {x}_{o} / {2}^{k} ] )} ) .$$

It is assumed that the standard deviation of the energy distribution of the initial state is small compared to the width of the spectrum of the Hamiltonian, which allows one to approximate the denominator in the above factor by Taylor expansion; one can see that the weight after measurement is suppressed as the distance

$$d = {min}_{n} \vert {2}^{k} ( E + n ) - {x}_{o} \vert$$

from the measurement outcome is increased, scaling with inverse squared

$$distance : ∼ \frac{1}{{d}^{2}} .$$

It is noted that the minimization over n in the definition of the distance is used as the expanded denominator is a periodic function.

If the precision of the coarse QPE and the postselection values are chosen appropriately, the high-energy weight of the distribution is suppressed.

### QETU Filtering

Using QETU (other Hamiltonian polynomial-based methods [17, 18] will work similarly) a polynomial function of the Hamiltonian that retains low energies and filters high energies is implemented.

In short, the method consists of a quantum signal processing circuit [19, 20] that implements a unitary matrix that block encodes a polynomial function f(H)=P(cos(H/2)), where H is the Hamiltonian of interest and P is an even polynomial of degree d. A scheme of the quantum circuit is shown in the schematic below. The circuit works by implementing U=e^{-iH} and its Hermitian conjugate controlled on a single ancilla a total number of d times.

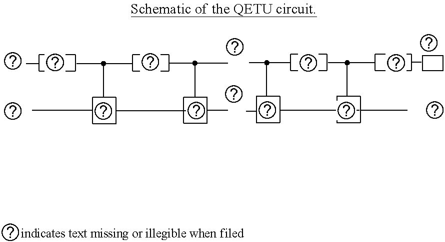

The parameters φ₀, φ₁, . . . , φ_{d/2} are determined based on the polynomial of interest. Upon measuring the ancilla qubit at the end and obtaining the outcome 0, the implementation has been successful. The probability of success is given by ∥P(cos(H/2))|ψ [symbol] ∥.

The polynomial that needs to be implemented for the energy filtering task should be a symmetric function that retains low energies and filters high energies. In particular, the spectrum of the Hamiltonian is chosen to lie within the interval [−π+η, 0−η]. If necessary, this can be done by adding a constant to and/or rescaling the Hamiltonian before performing QETU. It is noted that this is contrary to the original setting of Ref. [16] (the spectrum is contained in [η, π−η]) and coarse QPE mentioned above; the reason for this change is better performance as will become clear below. With this, low energies will be close to −π and thus cos(H/2) will be close to zero. As the goal is to retain low energies and filter out high energies, a polynomial P whose value is close to 1 when its argument is close to 0, and close to 0 when its argument is close to 1, is needed. First, the following combination of error functions is used to this end:

$${ξ}_{k , μ} ( x ) = \frac{1}{2} [ erf ( - k ( x - μ ) ) + erf ( k ( x + μ ) ) ]$$

with 0<k and 0<μ<1 determining the steepness and position of the transitions in the function. The prescription in Appendix A of Ref. [21] is used to reconstruct the error function erf(kx) in terms of Chebyshev polynomials as follows:

$${p}_{erf , k , n} = \frac{2 {ke}^{- {k}^{2} / 2}}{\sqrt{π}} ( {I}_{0} ( \frac{{k}^{2}}{2} ) + {\sum }_{j = 1}^{( n - 1 ) / 2} {( - 1 )}^{j} {I}_{j} ( {k}^{2} / 2 ) [ \frac{{T}_{2 j + 1} ( x )}{2 j + 1} - \frac{{T}_{2 j - 1} ( x )}{2 j - 1} ] )$$

where Tⱼ is the degree j Chebyshev polynomial and Iⱼ is the modified Bessel function of the first kind. It is noted that p_{erf,k,n} is an odd polynomial of degree n; it is the degree n that controls the error in approximating erf(kx) and thus ensures that low energies are retained and high energies are filtered, and it should therefore be chosen to be large enough.

Applying a successful round of QETU filtering to a state |ψ [symbol] =Σ_{E}φ_{E}|E [symbol] results in the following unnormalized state:

$${\sum }_{E} {φ}_{E} P ( cos ( E / 2 ) ) \vert E 〉 \vert 0 〉$$

This shows that, supposing one wishes to keep energies below Eₗ and filter energies above Eᵤ, one can choose a filtering function with

$$μ = \frac{cos ( {E}_{u} / 2 ) + cos ( {E}_{l} / 2 )}{2} and \frac{1}{k} = ζ \frac{cos ( {E}_{u} / 2 ) - cos ( {E}_{l} / 2 )}{2} .$$

The factor ζ is added so that it is possible to control the intensity of filtering while keeping the degree of the polynomial and the cost down.

The degree of the polynomial that needs to be used for this task will have a scaling of [symbol] (Γ⁻¹log ϵ⁻¹), where ϵ is the error in the polynomial approximation and Γ is the length scale over which the transition in the error functions in the equation for ϵ_{k,μ}(x) happens, and thus should scale as 1/k. In practice, n can be chosen by examining how good of an approximation is achieved for degree n.

### Costs of the Above Methods

To illustrate the advantages of the above methods (coarse QPE and QETU filtering) a simple analysis of the asymptotic cost is provided below by the following treatment: to suppress a high degree of weight at unwanted energies and keep low-energy weight mostly unsuppressed, one needs to differentiate energies at the level of the standard deviation of the energy distribution function of the initial state, which is shown as σ_{E}. The above two scenarios are considered separately.

In a coarse QPE setting, one needs the resolution to be of the order of σ_{E} at least, so that the unwanted weights are dropped when a low value outcome is postselected. This means that a number of queries to e^{-iH} is needed that scales as [symbol] (1/σ_{E}).

On the other hand, in a QETU setting, again a polynomial is needed that discerns energies that are both close to −π but differ by a value close to [symbol] (σ_{E}). After taking cos(H/2), one can use a Taylor expansion to see that the resolution is still [symbol] (σ_{E}) and thus a polynomial that transitions in a length scale scaling as [symbol] (σ_{E}) is needed. This causes the QETU approach to also have a scaling as [symbol] (1/σ_{E}). It is noted that if the spectrum had been chosen to lie in [η, π−η], the scaling would have been [symbol] (σ_{E}²).) as the required resolution after taking the cos function becomes [symbol] (σ_{E}²).

As both of the above methods give the same [symbol] (1/σ_{E}) scaling, this should in principle be a subleading cost when compared to the cost of the most precise QPE (or other energy estimation), which would have a scaling [symbol] (1/ϵ), as the precision e is expected to be considerably smaller than the width of the distribution given by σ_{E}.

It can be seen that the costs are asymptotically the same.

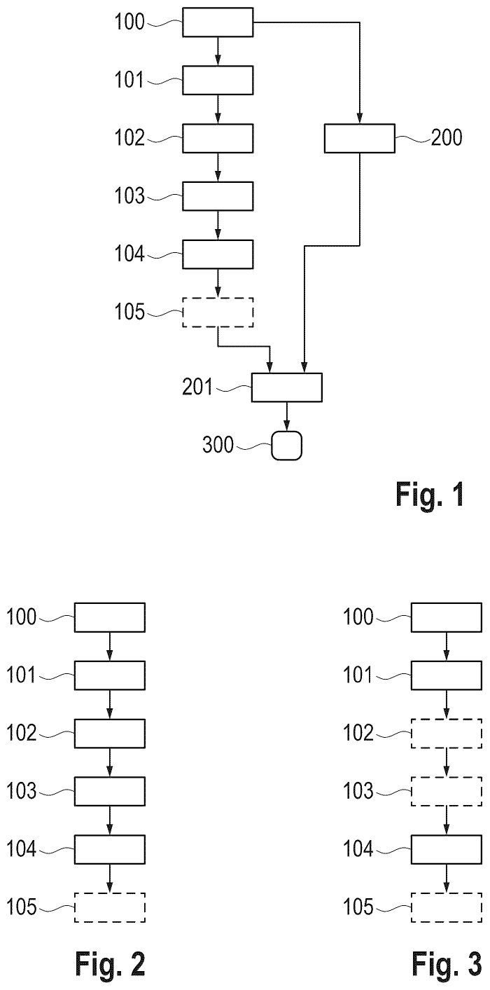

FIG. 1 shows a schematic diagram to illustrate embodiments of the method for estimating the ground state energy of a quantum chemical system.

In box 100, the Hamiltonian governing the physics of the quantum chemical system for which the ground state estimation should be performed is specified. There are a variety of systems of interest that may be considered, such as molecules, materials, and effective spin models. A variety of representations (e.g., first/second quantization) and approximations (e.g., pseudopotentials, rank factorization, quantum embedding) may be utilized to obtain the most suitable and efficient form of the Hamiltonian.

In box 101, with the Hamiltonian as the input, a classical computation is performed to obtain states close to the ground state of the quantum chemical system. The resulting states are a set of candidate states. The goal is to obtain an initial state that potentially has large support on the low-energy subspace of the Hamiltonian (e.g., the ground state). High overlap, for which high support is a proxy, is commonly a strict requirement for quantum algorithms to yield good estimates of the ground state energy. Different classical methods may be used, including Hartree-Fock, Coupled Cluster (CC), selective configuration interaction methods such as stochastic heat bath configuration space (SHCI), and density matrix renormalization group (DMRG).

In box 102, the quality of the classically computed states is assessed based on the energy distribution of each state. The different classical solutions (candidate states) are assessed by approximating the overlaps of the wavefunction with the Hamiltonian's true eigenstates, obtaining an approximate energy distribution of the state. This provides a means for quality comparison between different potential states.

Box 102 may involve approximating the energy distributions of at least a subset of the classically computed states through an Edgeworth series expansion using the Hamiltonian moments of the state and/or by the resolvent (matrix product state-based) method. The details for these methods are provided in the general description of this disclosure.

Alternatively or additionally, box 102 may further involve approximating the energy distributions of at least a subset of the classically computed states through coarse quantum phase estimation. The details for these methods are provided in the general description of this disclosure. It is noted that the classically computed states must be implemented on a quantum computer to perform coarse QPE. In box 103, a state is selected from the candidate states based on the assessment result. The support on the low-energy subspace is an important quantity to consider as it is proportional to the probability of obtaining energy estimates as low as those energies in a quantum routine. A state that has the largest weights for smaller energies is chosen as the best candidate. The details are provided in the general description.

In box 104, the selected state is implemented on a quantum computer. The classical state is encoded on the quantum computer. In other words, the selected state is encoded in a quantum register of the quantum computer.

Implementing the selected state in box 104 may involve preparing and using a compressed representation of the sum of Slater determinants. The details for these methods are provided in the general description of this disclosure.

Alternatively, implementing the selected state in box 104 may involve preparing and using a matrix product state form. The details for these methods are provided in the general description of this disclosure.

In an optional box 105, quantum refining of the implemented state on the quantum computer is performed by filtering out higher energy contributions after state implementation. The details for these methods are provided in the general description of this disclosure.

Quantum refining in box 105 may involve using at least one of the following: Hamiltonian polynomial methods, coarse quantum phase estimation, quantum imaginary time evolution, and quantum eigenvalue transformation of unitary matrices with real polynomials (QETU). The details for these methods are provided in the general description of this disclosure.

In box 200, the Hamiltonian of the system is encoded into the quantum computer. This is done by linear combination of unitaries (LCU), which may further be used for the qubitization of the system. In qubitization, a unitary operator Q (called the qubitization of H) is formed. Roughly speaking, the eigenstates of H are also eigenstates of Q, with eigenvalues related to those of H. Other methods, such as Hamiltonian simulation, may also be used.

In box 201, the ground state energy of the quantum chemical system is estimated on the quantum computer based on the implemented state. There are many methods to obtain the ground state energy estimate, which generally require an initial state input that has high enough support on the low-energy subspace of the system Hamiltonian. This state is derived in boxes 100 to 105. As the methods described in this disclosure provide an initial state with large overlap, the methods are applicable generally. Quantum phase estimation (QPE) is used for estimating the energy. The overlap between an initial state and a particular state of interest determines the number of times QPE measurements need to be performed as 1/overlap². Thus, better overlap, achieved by the embodiments of the method described in this disclosure, directly decreases the quantum computational cost.

The final result 300 is an improved estimate of the ground state energy of the Hamiltonian of the system.

FIG. 2 shows a schematic diagram to illustrate embodiments of the method for preparing a quantum state for quantum energy estimation on a quantum chemical system according to the second aspect.

In box 100, the Hamiltonian governing the physics of the quantum chemical system for which the ground state estimation should be performed is specified. There are various systems of interest that may be considered, such as molecules, materials, and effective spin models. Different representations (e.g., first/second quantization) and approximations (e.g., pseudopotentials, rank factorization, quantum embedding) may be utilized to obtain the most suitable and efficient form of the Hamiltonian.

In box 101, with the Hamiltonian as the input, a classical computation is performed to obtain states close to the ground state of the quantum chemical system. The resulting states are a set of candidate states. The goal is to obtain an initial state that potentially has a large overlap with the lowest-energy eigenstates of the Hamiltonian (e.g., the ground state). High overlap, for which support is a proxy, is commonly a strict requirement for quantum algorithms to yield good estimates of the ground state energy. Different classical methods may be used, such as Hartree-Fock, Coupled Cluster (CC), selective configuration interaction methods (such as stochastic heat bath configuration space, SHCI), and density matrix renormalization group (DMRG).

In box 102, the quality of the classically computed states is assessed based on the energy distribution of each state. The different classical solutions (candidate states) are assessed by approximating the overlaps of the wavefunction with the Hamiltonian's true eigenstates, obtaining an approximate energy distribution of the state. This provides a means for quality comparison between different potential states.

Box 102 may involve approximating the energy distributions of at least a subset of the classically computed states through an Edgeworth series expansion using the Hamiltonian moments of the state and/or by the resolvent (matrix product state-based) method. The details for these methods are provided in the general description of this disclosure.

Alternatively or additionally, box 102 may further involve approximating the energy distributions of at least a subset of the classically computed states through coarse quantum phase estimation. The details for these methods are provided in the general description of this disclosure. It is noted that the classically computed states must be implemented on a quantum computer to perform coarse QPE.

In box 103, a state is selected from the candidate states based on the assessment result. To choose a state from the candidate states as a solution for the initial state to start with, the support on the low-energy subspace is an important quantity to consider, as it is proportional to the probability of obtaining energy estimates as low as those energies in a quantum routine. A state that has the largest weights for smaller energies is chosen as the best candidate. The details are provided in the general description.

In box 104, the selected state is implemented on a quantum computer. The classical state is encoded on the quantum computer, meaning the selected state is encoded in a quantum register of the quantum computer.

Implementing the selected state in box 104 may involve preparing and using a compressed representation of the sum of Slater determinants. The details for these methods are provided in the general description of this disclosure.

Alternatively, implementing the selected state in box 104 may involve preparing and using a matrix product state form. The details for these methods are provided in the general description of this disclosure.

In an optional box 105, quantum refining of the implemented state on the quantum computer is performed by filtering out higher energy contributions after state implementation. The details for these methods are provided in the general description of this disclosure.

Quantum refining in box 105 may involve using at least one of the following: Hamiltonian polynomial methods, coarse quantum phase estimation, quantum imaginary time evolution, and quantum eigenvalue transformation of unitary matrices with real polynomials (QETU). The details for these methods are provided in the general description of this disclosure.

FIG. 3 shows a schematic diagram to illustrate embodiments of the method for preparing a quantum state for quantum energy estimation on a quantum chemical system according to the third aspect.

In box 100, the Hamiltonian governing the physics of the quantum chemical system for which the ground state estimation should be performed is specified. There are various systems of interest that may be considered, such as molecules, materials, and effective spin models. Different representations (e.g., first/second quantization) and approximations (e.g., pseudopotentials, rank factorization, quantum embedding) may be utilized to obtain the most suitable and efficient form of the Hamiltonian.

In box 101, with the Hamiltonian as the input, a classical computation is performed to obtain a state close to the ground state of the quantum chemical system. The goal is to obtain an initial state that potentially has a large overlap with the lowest-energy eigenstates of the Hamiltonian (e.g., the ground state). High overlap, for which support is a proxy, is commonly a strict requirement for quantum algorithms to yield good estimates of the ground state energy. Different classical methods may be used, such as Hartree-Fock, Coupled Cluster (CC), selective configuration interaction methods (such as stochastic heat bath configuration space, SHCI), and density matrix renormalization group (DMRG).

Optionally, in box 101, classically computing the state may involve classically computing a plurality of states close to the ground state of the quantum chemical system.

In optional box 102, the quality of the classically computed states is assessed based on the energy distribution of each state. The different classical solutions (candidate states) are assessed by approximating the overlaps of the wavefunction with the Hamiltonian's true eigenstates, obtaining an approximate energy distribution of the state. This provides a means for quality comparison between different potential states.

Optional box 102 may involve approximating the energy distributions of at least a subset of the classically computed states through an Edgeworth series expansion using the Hamiltonian moments of the state and/or by the resolvent (matrix product state- based) method. The details for these methods are provided in the general description of this disclosure.

Alternatively or additionally, optional box 102 may further involve approximating the energy distributions of at least a subset of the classically computed states through coarse quantum phase estimation. The details for these methods are provided in the general description of this disclosure. It is noted that the classically computed states must be implemented on a quantum computer to perform coarse QPE.

In optional box 103, a state is selected from the candidate states based on the assessment result. To choose a state from the candidate states as a solution for the initial state to start with, the support on the low-energy subspace is an important quantity to consider, as it is proportional to the probability of obtaining energy estimates as low as those energies in a quantum routine. A state that has the largest weights for smaller energies is chosen as the best candidate. The details are provided in the general description.

In box 104, the (optionally selected) state is implemented on a quantum computer. The classical state is encoded on the quantum computer, meaning the selected state is encoded in a quantum register of the quantum computer.

Implementing the selected state in box 104 involves preparing and using a compressed representation of the sum of Slater determinants. The details for these methods are provided in the general description of this disclosure.

In an optional box 105, quantum refining of the implemented state on the quantum computer is performed by filtering out higher energy contributions after state implementation. The details for these methods are provided in the general description of this disclosure.

Quantum refining in box 105 may involve using at least one of the following: Hamiltonian polynomial methods, coarse quantum phase estimation, quantum imaginary time evolution, and quantum eigenvalue transformation of unitary matrices with real polynomials (QETU). The details for these methods are provided in the general description of this disclosure.

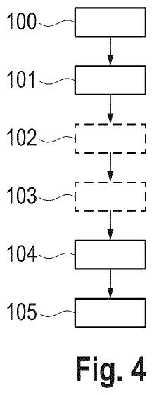

FIG. 4 shows a schematic diagram to illustrate embodiments of the method for preparing a quantum state for quantum energy estimation on a quantum chemical system according to the fourth aspect.

In box 100, the Hamiltonian governing the physics of the quantum chemical system for which the ground state estimation should be performed is specified. There are various systems of interest that may be considered, such as molecules, materials, and effective spin models. Different representations (e.g., first/second quantization) and approximations (e.g., pseudopotentials, rank factorization, quantum embedding) may be utilized to obtain the most suitable and efficient form of the Hamiltonian.

In box 101, with the Hamiltonian as the input, a classical computation is performed to obtain a state close to the ground state of the quantum chemical system. The goal is to obtain an initial state that potentially has a large overlap with the lowest-energy eigenstates of the Hamiltonian (e.g., the ground state). High overlap, for which support is a proxy, is commonly a strict requirement for quantum algorithms to yield good estimates of the ground state energy. Different classical methods may be used, such as Hartree-Fock, Coupled Cluster (CC), selective configuration interaction methods (such as stochastic heat bath configuration space, SHCI), and density matrix renormalization group (DMRG).

Optionally, in box 101, classically computing the state may involve classically computing a plurality of states close to the ground state of the quantum chemical system.

In optional box 102, the quality of the classically computed states is assessed based on the energy distribution of each state. The different classical solutions (candidate states) are assessed by approximating the overlaps of the wavefunction with the Hamiltonian's true eigenstates, obtaining an approximate energy distribution of the state. This provides a means for quality comparison between different potential states.

Optional box 102 may involve approximating the energy distributions of at least a subset of the classically computed states through an Edgeworth series expansion using the Hamiltonian moments of the state and/or by the resolvent (matrix product state- based) method. The details for these methods are provided in the general description of this disclosure.

Alternatively or additionally, optional box 102 may further involve approximating the energy distributions of at least a subset of the classically computed states through coarse quantum phase estimation. The details for these methods are provided in the general description of this disclosure. It is noted that the classically computed states must be implemented on a quantum computer to perform coarse QPE.

In optional box 103, a state is selected from the candidate states based on the assessment result. To choose a state from the candidate states as a solution for the initial state to start with, the support on the low-energy subspace is an important quantity to consider, as it is proportional to the probability of obtaining energy estimates as low as those energies in a quantum routine. A state that has the largest weights for smaller energies is chosen as the best candidate. The details are provided in the general description.

In box 104, the (optionally selected) state is implemented on a quantum computer. The classical state is encoded on the quantum computer, meaning the selected state is encoded in a quantum register of the quantum computer.

Implementing the selected state in box 104 may involve preparing and using a compressed representation of the sum of Slater determinants. The details for these methods are provided in the general description of this disclosure.

Alternatively, implementing the selected state in box 104 may involve preparing and using a matrix product state form. The details for these methods are provided in the general description of this disclosure.

In box 105, quantum refining of the implemented state on the quantum computer is performed by filtering out higher energy contributions after state implementation. The details for these methods are provided in the general description of this disclosure.

Quantum refining in box 105 may involve using at least one of the following: Hamiltonian polynomial methods, coarse quantum phase estimation, quantum imaginary time evolution, and quantum eigenvalue transformation of unitary matrices with real polynomials (QETU). The details for these methods are provided in the general description of this disclosure.

The methods shown in FIGS. 2, 3, and 4 may all be used within a method for estimating the ground state energy of a quantum chemical system as shown in FIG. 1 and described above. Every individual feature of the embodiments in this disclosure may be combined with other features in any combination. Any variant of a combination of at least one of assessing the quality, implementing the state by preparing and using a compressed representation of Slater determinants, and quantum refining may be used.

Any of the methods described may further comprise categorizing the ground state energy estimation problem of the quantum chemical system with regard to computational difficulty as either an easy problem, an intermediate problem, or a hard problem, and only if the result shows an intermediate problem, performing all method steps.

Any of the methods described may further comprise calculating, based on the energy distribution, the mean value of the distribution of the smallest energy obtained from within a quantum phase estimation budget of N outcomes, assessing whether it is below a target classical energy, and only if this is the case, performing all method steps.

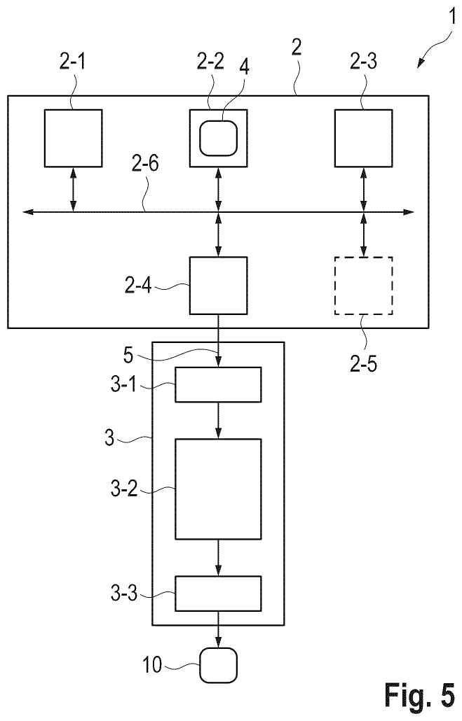

FIG. 5 shows a schematic of an embodiment of the system 1. The system 1 comprises a classical computer 2 and a quantum computer 3.

The classical computer 2 comprises at least one processor 2-1, at least one memory 2-2, a persistent storage 2-3, a communication interface 2-4, and optionally a display 2-5. These components 2-1, 2-2, 2-3, 2-4, and 2-5 are connected via at least one communication bus 2-6. The classical computer is configured to perform the classical computations of a method according to any one of the embodiments described above.

The quantum computer 3 comprises at least one input quantum register 3-1, a quantum circuit 3-2, and an output register 3-3. The quantum circuit 3-2 comprises quantum gates that act upon a state input to the input quantum register 3-1. The quantum computer 3 is configured to perform the quantum computations of the embodiment. The initial state derived by the classical computation is implemented in the quantum input register 3-1 and then propagated through the quantum circuit 3-2. The output register 3-3 then holds the result. To obtain the result, a number of QPE measurements are necessary. In the last phase of the QPE, the quantum computer 3 performs measurements on the qubits to extract the desired information. This process is repeated several times to produce the chemically accurate prediction of the ground state energy.

The quantum computer 3 may be composed of superconducting qubits, photonic qubits, trapped-ion qubits, silicon-based qubits, and/or neutral atoms.

The methods and system described in this disclosure decrease the cost of energy estimation, particularly in the QPE algorithm. They decrease the number of times that the final most accurate and most costly QPE needs to be performed by preparing a state with larger support on the low-energy subspace of the Hamiltonian. By increasing the overlap, there is also the potential to make each QPE measurement less costly. In some cases, due to a poor overlap, the total QPE evolution time might need to be as long as [symbol] (1/overlap²); with a better overlap, achievable by using the methods and the system described in this disclosure, the total time can be shortened and can essentially be independent of the overlap. This is due to preventing leakage from high-energy states and thus decreasing the total evolution time required for each QPE round.

### APPENDIX A

In general, the Edgeworth series terms can be written as [12]:

$${p}_{E} ( x ) = \frac{{e}^{- {x}^{2} / 2}}{\sqrt{2 π}} [ 1 + {\sum }_{s = 1}^{∞} {\sum }_{\{ {k}_{m} \}} H {e}_{s + 2 r} ( x ) {\prod }_{m = 1}^{s} \frac{1}{{k}_{m} !} {( \frac{{κ}_{m + 2}}{( m + 2 ) !} )}^{{k}_{m}} ]$$

where the summation over {kₘ} in the above denotes summation over all non-negative integer solutions of the Diophantine equation

$${k}_{1} + 2 {k}_{2} + … + s {k}_{s} = s$$

and r is the sum of these integers for each solution: r=Σkₘ.

### APPENDIX B

### MPS to Sum of Slater Determinants

The goal is to calculate the largest coefficients c(n₁, . . . , n_{N})=Σ_{α}_{₁} . . . . α_{N-1}A_{a;α}_{ₐ}ⁿ^{₁} . . . A_{N;α}_{_{N−1}}ⁿ^{_{N}} in a sum of Slater determinants (SOS) expansion. Based on Appendix A of Ref. [7], one starts from a left canonical form and sets a threshold for keeping terms in the SOS. Partial coefficients such as c_{α}_{ₚ}^{(p)}(n₁, . . . , nₚ)=Σ_{α}_{₁} . . . . αₚ₋₁A_{a;α}_{ₐ}ⁿ^{₁} . . . A_{p;α}_{_{p−1}}ⁿ^{ₚ} are formed and whenever a norm of the partial coefficient Σ₆₀ _{^{p}}|c_{α}_{ₚ}^{(p)}(n₁, . . . , nₚ)|² goes below a threshold, all Slater determinants with the prefix (n₁, . . . , nₚ), i.e., of the form |n₁, . . . , nₚ, . . . [symbol] , are dropped from the SOS. This way, owing to the left canonical form of the MPS, it is ensured that all the terms with coefficients above the threshold are recovered in the SOS.

### Sum of Slater Determinants to MPS

For this task, one starts with a bond dimension 1 MPS that corresponds to the largest coefficient Slater determinant in the SOS (could be the Hartree-Fock state or not); one also makes an auxiliary copy of it. Using matrix product operators consisting of a number of c† and c operators, the auxiliary bond dimension 1 MPS is transformed to the Slater determinant with the second largest coefficient. The new auxiliary MPS is added to the main MPS and the procedure goes on until all coefficients are added. It is noted that the auxiliary MPS remains bond dimension 1 while the bond dimension of the main MPS grows, and one can compress the main MPS as more and more terms are added to it.

### REFERENCES

[1] A. Y. Kitaev, arXiv preprint quant-ph/9511026 (1995).
[2] M. A. Nielsen and I. L. Chuang, Quantum computation and quantum information (Cambridge university press, 2010).
[3] N. M. Tubman, C. Mejuto-Zaera, J. M. Epstein, D. Hait, D. S. Levine, W. Huggins, Z. Jiang, J. R. McClean, R. Babbush, M. Head-Gordon, et al., arXiv preprint arXiv:1809.05523 (2018).
[4] C. Schön, E. Solano, F. Verstraete, J. I. Cirac, and M. M. Wolf, Physical review letters 95, 110503 (2005).
[5] D. Malz, G. Styliaris, Z.-Y. Wei, and J. I. Cirac, arXiv preprint arXiv:2307.01696 (2023).
[6] H. Zhai and G. K. Chan, The Journal of Chemical Physics 154 (2021).
[7] S. Lee, H. Zhai, S. Sharma, C. J. Umrigar, and G. K.-L. Chan, Journal of Chemical Theory and Computation 17, 3414 (2021).
[8] G. H. Low, V. Kliuchnikov, and L. Schaeffer, arXiv preprint arXiv:1812.00954 (2018).
[9] S.-J. Ran, Physical Review A 101, 032310 (2020). M. S. Rudolph, J. Chen, J. Miller, A. Acharya, and A. Perdomo-Ortiz, arXiv preprint arXiv:2209.00595 (2022).
[11] M. B. Dov, D. Shnaiderov, A. Makmal, and E. G. D. Torre, arXiv preprint arXiv:2207.00028 (2022).
[12] S. Blinnikov and R. Moessner, Astronomy and Astrophysics Supplement Series 130, 193 (1998).
[13] E. Jeckelmann, Physical Review B 66, 045114 (2002).
[14] E. Ronca, Z. Li, C. A. Jimenez-Hoyos, and G. K.-L. Chan, Journal of chemical theory and computation 13, 5560 (2017).
[15] M. Wand and M. Jones, Kernel Smoothing, Chapman & Hall/CRC Monographs on Statistics & Applied Probability (60) (Chapman & Hall/CRC, 1994).
[16] Y. Dong, L. Lin, and Y. Tong, PRX Quantum 3, 040305 (2022).
[17] L. Lin and Y. Tong, Quantum 4, 372 (2020).
[18] Y. Ge, J. Tura, and J. I. Cirac, Journal of Mathematical Physics 60 (2019).
[19] G. H. Low and I. L. Chuang, Physical review letters 118, 010501 (2017).
[20] J. M. Martyn, Z. M. Rossi, A. K. Tan, and I. L. Chuang, PRX Quantum 2, 040203 (2021).
[21] G. H. Low and I. L. Chuang, arXiv preprint arXiv:1707.05391 (2017).
[22] |22| L. Lin and Y. Tong, PRX Quantum 3, 010318 (2022).
[23] Y. Ge, J. Tura, and J. I. Chirac, Journal of Mathematical Physics 60 (2019).

### REFERENCE NUMERALS

1 system
2 classical computer
2-1 processor
2-2 memory
2-3 persistent storage
2-4 communication interface
2-5 display
2-6 communication bus
3 quantum computer
3-1 input quantum register
3-2 quantum circuit
3-3 output register
4 state
5 selected/implemented state
100-105 method boxes
200-201 method boxes
300 final result

1. A method for estimating a ground state energy of a quantum chemical system, the method comprising:
classically computing states close to the ground state of the quantum chemical system;
assessing the quality of the classically computed states based on the energy distribution of each state;
selecting a state based on the assessment result;
implementing the selected state on a quantum computer; and
estimating the ground state energy of the quantum chemical system on the quantum computer based on the implemented state.

2. The method of claim 1, further comprising approximating the energy distributions of at least a subset of the classically computed states through an Edgeworth series expansion using the Hamiltonian moments of the state and/or by the resolvent method.

3. The method of claim 2, further comprising approximating the energy distributions of at least a subset of the classically computed states through coarse quantum phase estimation.

4. The method of claim 1, further comprising implementing the selected state by preparing and using a compressed representation of the sum of Slater determinants.

5. The method of claim 1, further comprising implementing the selected state by preparing and using a matrix product state form.

6. The method of claim 1, further comprising quantum refining of the implemented state on the quantum computer by filtering out higher energy contributions after state implementation.

7. The method of claim 6, wherein the quantum refining comprises using at least one of Hamiltonian polynomial methods, coarse quantum phase estimation, and quantum

8. A method for preparing a quantum state for quantum energy estimation on a quantum chemical system, the method comprising:
classically computing a state close to the ground state of the quantum chemical system;
implementing the state on a quantum computer; and
refining the implemented state on the quantum computer by filtering out higher energy contributions after state implementation.

9. The method of claim 8, wherein classically computing the state comprises classically computing a plurality of states close to the ground state of the quantum chemical system, assessing the quality of the classically computed states based on the energy distribution of each state, and selecting a state based on the assessment result for implementing.

10. The method of claim 8, further comprising categorizing the ground state energy estimation problem of the quantum chemical system with regard to computational difficulty as either an easy problem, an intermediate problem, or a hard problem, and only if the result shows an intermediate problem, performing all method steps.

11. The method of claim 8, further comprising approximating the energy distributions of at least a subset of the classically computed states through an Edgeworth series expansion using the Hamiltonian moments of the state and/or by the resolvent method.

12. The method of claim 11, further comprising approximating the energy distributions of at least a subset of the classically computed states through coarse quantum phase estimation.

13. The method of claim 8, wherein implementing the selected state comprises preparing and using a compressed representation of the sum of Slater determinants.

14. A system comprising:
a classical computer configured to:
classically compute states close to the ground state of the quantum chemical system;
assess the quality of the classically computed states based on the energy distribution of each state;
select a state based on the assessment result; and
a quantum computer configured to:
implement the selected state; and
estimate the ground state energy of the quantum chemical system based on the implemented state.

15. The system of claim 14, wherein the classical computer is further configured to approximate the energy distributions of at least a subset of the classically computed states through an Edgeworth series expansion using the Hamiltonian moments of the state and/or by the resolvent method.

16. The system of claim 15, wherein the classical computer is further configured to approximate the energy distributions of at least a subset of the classically computed states through coarse quantum phase estimation.

17. The system of claim 14, wherein the quantum computer is further configured to implement the selected state by preparing and using a compressed representation of the sum of Slater determinants.

18. The system of claim 14, wherein the quantum computer is further configured to implement the selected state by preparing and using a matrix product state form.

19. The system of claim 14, wherein the quantum computer is further configured to perform quantum refining of the implemented state by filtering out higher energy contributions after state implementation.

20. The system of claim 19, wherein the quantum refining comprises using at least one of: Hamiltonian polynomial methods, coarse quantum phase estimation, and quantum
